(19) United States
(12) Patent Application Publication
(10) Pub. No.: US 2011/0207162 A1
(43) Pub. Date: Aug. 25, 2011

(54) Dynamic visualization of expressed gene networks in living cells

(75) Inventors: Stephen William Watson Michnick, Montreal (CA); Ingrid Remy, Montreal (CA)

(73) Assignee: Odyssey Thera, Inc., San Ramon, CA (US)

(21) Appl. No.: 12/923,256

(22) Filed: Sep. 10, 2010

Related U.S. Application Data
(63) Continuation of application No. 09/968,864, filed on Oct. 3, 2001, now abandoned.

(30) Foreign Application Priority Data
Jan. 31, 1997 (CA) 2,196,146

(51) Int. Cl. C12Q 1/02

(52) U.S. Cl. 435/29

(57) ABSTRACT

The present invention provides functional annotation of novel genes by detection of interactions of their encoded proteins with known proteins followed by assays to validate that the gene participates in a specific cellular function. The instant invention also provides an experimental strategy that allows for detection of protein interactions and functional assays with a single reporter system. Interactions among network component proteins are detected and probed with stimulators and inhibitors of the network and subcellular location of the interacting proteins is determined. Additionally, applicants' use this strategy to map a signal transduction network that controls the G₀ to G₁ transition in eukaryotes. Analysis of 148 combinations of 65 protein pairs in mammalian cells allows applicants' to propose a model of network architecture. The results demonstrate the feasibility of employing this strategy in genome-wide functional annotation efforts.

### BACKGROUND OF THE INVENTION

Rapid progress in genome projects is leading to the identification or prediction of a huge number of genes, but only a fraction of gene functions can be inferred from primary gene sequences. A first step in defining the function of a gene is to determine its interactions with other gene products. This is the basis of the highly successful Yeast Two-Hybrid system (1, 2). The second step is to perform functional assays in model cells or whole organisms from which the genes in question were derived. It would be advantageous if one could combine screening of protein-protein interactions with tests for biological relevance using a single assay system, thus validating the screening results and eliminating spurious interactions immediately. Therefore, we developed (See U.S. Pat. No. 6,270,964 B1) an experimental approach for detecting protein-protein interactions in intact living cells based on protein interaction-induced folding and reconstitution of activity of the enzyme murine dihydrofolate reductase (DHFR) from two rationally-dissected fragments of the enzyme (3-6). Generally we call this, and other assays we have developed based on the same principle, Protein fragment Complementation Assays (PCA) (6). We have demonstrated that the DHFR PCA can be used as a sensitive survival selection assay and also as a fluorescence assay that allows for quantitative detection of induced protein-protein interactions (4, 5). Here, we describe a strategy and a proof of principle for the use of the DHFR PCA in functional validation of protein interactions and for the mapping of biochemical pathways.

In the course of the present discoveries, applicants' needed to answer the following question: If we observe an interaction between two proteins in a simple screen (survival-selection assay), what additional information must be generated—for example, using the fluorescence assay—to show that the interaction is biologically relevant? FIG. 1A provides a scheme for understanding the organization of proteins within a biochemical and specifically a signal transduction pathway.

Signal transduction pathways have proven useful models for examining the organization of biochemical pathways and networks for the following reasons. First, signal transduction pathways have defined initiation events, such as hormone activation of a membrane receptor, and thus in principle it is straightforward to define the quiescent and stimulated states of the pathway. Second, the position of a specific interaction within a series of events in a signaling cascade can be determined by examining the effects of agents that inhibit or activate known steps in the pathway. The agents could include specific small molecule inhibitors or activators, dominant-negative mutants of biochemical pathway component proteins, or anti-sense RNA for individual components of the pathway. Observing induction and inhibition of individual protein-protein interactions in a signaling cascade would provide functional validation in the following ways: (1) Perturbation of protein-protein interactions in predicted ways by hormones and inhibitors would provide convincing evidence that the proteins participate in the pathway; (2) The way in which the stimulators and inhibitors or activators affect an interaction (the “pharmacological profile”) would provide evidence for the position of the particular interaction within the pathway; (3) Signal transduction pathways are hierarchically organized in space, with early events occurring at the inner membrane surface while later events may occur in the cytosol, nucleus or other subcellular compartments. A biologically relevant protein-protein interaction must occur at a surface or within a cellular compartment that is consistent with its position within the signaling cascade; and (4) Novel pharmaceutical targets are those proteins which play an active role in a disease-related pathway, that is, a pathway which is associated with or perturbed in the disease state, for which either there are agents with known therapeutic efficacy or for which chemical inhibitors can be developed.

The present invention provides that screening, pharmacological profiling and the cellular location of protein-protein interactions can be achieved using the survival-selection and fluorescence DHFR PCA. In the course of the present invention, we provide the signal transduction pathway involved in insulin and growth factor receptor tyrosine kinase (RTK)-mediated control of translation-initiation in eukaryotes (FIG. 1B). We also provide a parallel pathway that both cross-talks with the RTK pathway and has affects on proteins involved with translation-initiation that converge with the RTK pathway. This pathway is controlled by the serine/threonine kinase FRAP (7, 8).

The prior art is silent on the use of the DHFR PCA in functional validation of protein interactions, mapping of biochemical pathways and identification of pharmaceutical targets.

### SUMMARY OF THE INVENTION

The present invention provides a method of testing at least two molecules for possible mutual interaction comprising causing a cell to contain said molecules, each of which is coupled to a separate part of a reporter system, which parts directly or indirectly provide a detectable signal upon their association, and testing for the presence of said signal.

The invention also provides a method of identifying at least two molecules capable of mutual interaction that may function as members of a biochemical pathway comprising causing a cell which expresses said pathway to contain said molecules, each of which is coupled to a separate part of a reporter system, which parts directly or indirectly provide a detectable signal upon their association, testing for said signal, and identifying said molecules, when signal is present, as possible members of said pathway.

The instant invention also provides a method of determining whether two or more molecules are able to mutually interact in a cell comprising coupling said molecules to separate parts of a reporter system, which parts directly or indirectly provide a detectable signal upon their association, testing for said signal, and correlating said signal, when present, with the ability of said molecules to mutually interact in said cell.

The invention also describes a method of determining whether two or more molecules thought to participate intracellularly in a biochemical pathway are capable of mutual interaction within a cell suspected of expressing said pathway comprising forming a coupling product for each molecule, said coupling product comprising each molecule coupled to part of a reporter system, which parts directly or indirectly provide a detectable signal upon their association, causing said coupling products to be contained by said cell, testing for said signal, and designating said signal, when present, as evidence that said molecules interact intracellularly in said biochemical pathway.

Additionally, the invention is directed to a method for defining a model that describes how two or more cellular biochemical pathways are organized comprising: (1) identifying molecules known or suspected of being part of any of said pathways; (2) performing PCA assays to determine: (A) which of said molecules can mutually interact; (B) where in a cell such interactions can occur; and (C) how said interactions can be affected by stimulators or inhibitors of any of said pathways; and (3) defining a model which is consistent with all data gathered.

The invention further provides a method of determining a subcellular location where two or more molecules interact comprising coupling each molecule to a separate part of a reporter system, which parts directly or indirectly provide a detectable signal upon their association, and testing for the subcellular location of said signal.

The invention is also directed to a method of mapping a biochemical pathway comprising: (A) using PCA to determine the mutual interaction of at least a first and a second molecule present within a cell; (B) adding a compound or composition to said cell, or a clone thereof, to determine whether stimulation or inhibition of said mutual interaction occurs; (C) determining the subcellular location of said mutual interaction; and (D) creating a map of said biochemical pathway using the information gathered from (A), (B), and (C).

The invention is also directed to a method of identifying novel pharmaceutical targets comprising (A) using PCA to identify a protein that interacts with other proteins within a biochemical pathway; (B) validating that said protein actively participates in the pathway, by creating a pharmacological profile of the interaction and comparing the profile for that interaction with the profiles of other interactions in the same pathway; and (C) Based on the pharmacological profile and subcellular localization data, establishing a link between said protein and the pathway effects and/or phenotypic effects of a known or candidate therapeutic agent.

The invention is also directed to a method of identifying the site of action of a drug within a biochemical pathway, comprising (A) Introducing the drug into cells containing one or more biochemical pathways; (B) Establishing quantitative pharmacological profiles, e.g. using PCA, for specific protein-protein interactions within one or more biochemical pathways, in the absence and presence of drug; (C) Based on the pharmacological profiles, identifying the steps at which the drug activates or inhibits the pathway.

The invention is also directed to a method of screening combinatorial or natural product libraries to identify activators or inhibitors of specific steps within biochemical pathways, such method comprising (A) using PCA to construct an assay for one or more steps in a biochemical pathway; (B) testing the effects of compounds, e.g. from a compound library, on the protein or pathway(s) of interest; (C) Using the results of the screen to identify specific compounds which activate or inhibit the protein or pathway(s) of interest.

### DESCRIPTION OF THE FIGURES

FIG. 1(A) illustrates a schematic representation of the strategy for generating a functional validation profile of a biochemical network using the DHFR PCA. Positive clones are detected with the DHFR survival-selection assay. They correspond to interacting component proteins of two convergent signal transduction pathways (Path 1 and Path 2). An interaction matrix (upper left) represents all positive (green) and negative (red) interacting pairs observed in the survival-selection assay. Positive clones from survival selection are propagated and subjected to two functional analyses: 1) Using the DHFR fluorescence assay, interactions are probed with pathway specific stimulators (1 and 2) and inhibitors (A and B). Pharmacological profiles are established based on the pattern of response of individual interactions to stimulators and inhibitors, represented in the histograms (ordinate axis represent fluorescence intensity). For example, stimulation of pathway 1 will augment all the interactions composing that pathway. The inhibitor A will inhibit protein interactions downstream, but not upstream of its site of action in pathway 1. 2) Cellular locations of the interactions are determined by fluorescence microscopy, also using the DHFR fluorescence assay.

FIG. 1(B) describes the well established connections within RTK (growth factor activated) and FRAP mediated pathways that control translation-initiation and sites of action of inhibitors or activators of these pathways. Broken line indicates that action is indirect.

FIG. 2 shows the summary of the results obtained for the different protein-protein interactions tested in the RTK/FRAP network with the DHFR survival selection assay in CHO DUKX-B11 (DHFR⁻) cells. On the X axis are the fusions to DHFR[1,2] fragment and on the Y axis the fusions to DHFR[3] fragment. The orientations of the fusions (N-terminal or C-terminal) are also indicated. Positive interactions: green (+), absence of interaction: red (−), not tested: gray squares.

FIG. 3 describes the fluorometric and microscopic analysis of the wortmannin-sensitive/rapamycin-resistant components of the network. The grid represents all positive (green) and negative (red) interactions observed in survival-selection. The pharmacological profiles are represented by the histograms (right). Cells were treated with stimulants and inhibitors as described in “Example 3” (x-axis, NT=No Treatment, I=Insulin, S=Serum, R=Rapamycin, W=Wortmannin). Fluorescence intensity is given in relative fluorescence units (y-axis). The background fluorescence intensity corresponding to non-transfected cells was subtracted from the fluorescence intensities of all the samples. Error bars represent standard errors for the mean calculated from at least three independent experiments. Microscopy revealing patterns of locations is also presented. The dimerization of GCN4 leucine zipper (GCN4/GCN4) is used as a control. The fusion protein pairs used in these experiments were: PDK1-F[1,2]/PKB-F[3], F[1,2]-p70S6K/PKB-F[3], F[1,2]-FRAP/PKB-F[3], F[1,2]-p70S6K/PDK1-F[3], PDK1-F[1,2]/F[3]-FRAP, F[1,2]-FRAP/F[3]-FRAP, F[1,2]-FRAP/4EBP1-F[3], F[1,2]-GCN4/GCN4-F[3].

FIG. 4 illustrates the fluorometric and microscopic analysis of the rapamycin-sensitive components of the network (C=Calyculin A, all other abbreviations same as FIG. 3). The fusion protein pairs used in these experiments were: PP2A-F[1,2]/PKB-F[3], PP2A-F[1,2]/p70S6K-F[3], F[1,2]-FRAP/FKBP-F[3], F[1,2]-Cdc42/P70S6K-F[3], F[1,2]-Rac1/p70S6K-F[3], F[1,2]-p70S6K/S6-F[3], F[1,2]-p70S6K/4EBP1-F[3].

FIG. 5 is a summary of the protein-protein interactions involved in the RTK/FRAP network and their responses to inhibitors. This summary is based on the results obtained from survival-selection and pharmacological profile experiments. The effects of each drug are indicated (C=calyculin A, R=rapamycin, W=Wortmannin). The blue arrows indicate novel protein-protein interactions. Single head arrows indicate kinase or phosphatase-substrate catalytic events. Double head arrows represent equilibrium non-catalytic interactions. Broken arrows indicate that the nature of the interactions is unknown.

FIG. 6 (Gene to Target) illustrates a method, as a series of steps, for identifying and validating pharmaceutical targets, starting with the gene (X). The first step is to screen for interactions between the product of gene X and the members of one or more pathways, depicted as a gene-by-gene matrix (in practice, the screening can be performed using a gene-by-gene or a gene-by-library approach where the library is a cDNA library). Positive interactions between gene products are shown in green; lack of an interaction is depicted in red. Proteins interacting with other members of a pathway are then validated by constructing quantitative pharmacological profiles specific to the pathway in question, which is done by measuring the effects of perturbants (activators and inhibitors) on the protein-protein complexes and by determining the subcellular localization of the complexes e.g. using PCA. Proteins that actively participate in disease-related pathways are potential new targets for discovery of small-molecule, natural product or other potential drug molecules.

FIG. 7 (Drug to Target) illustrates a method, as a series of steps, for pinpointing within biochemical pathways the molecular target of a drug. Arrays representing individual steps in pathways are constructed and the effects of the drug are tested using quantitative assays e.g. using PCA. The target of the drug and its downstream effects can be determined by assessing each step in a pathway in the absence and presence of the drug of interest, and measuring the step(s) at which the drug causes inhibition.

FIG. 8 (Gene to Drug) illustrates a method for identifying drugs that inhibit biochemical pathways or individual steps within a pathway. The method involves testing compounds, such as small molecule or natural product libraries, against individual steps within a pathway or against entire pathways (represented as components of a gene-by-gene matrix) in order to detect compounds capable of inhibiting or inducing the formation of complexes within pathways.

### DESCRIPTION OF THE PREFERRED EMBODIMENTS

The present invention provides methodology for the quantitative analysis and profiling of induceable cellular responses for the identification of novel therapeutic targets assessing drug action and screening assays derived therefrom for discovery of novel modulators of cellular responses.

Cellular phenotypes and responses are mediated by a complex array of proteins that are resident within subcellular compartments. The particularly cellular protein architecture is controlled by regulatory elements within the encoded genetic information within the cell nucleus. Binding of proteins, amino acids, growth factors, hormones and other external stimuli such as UV irradiation or oxidative stress induce a cascade of intracellular interactions mediated initially by receptors or other signaling molecules within or associated with the cell membrane or in the cytoplasm. The regulation of cellular responses is mediated by a certain “threshold” number of molecular interactions that eventually reach the cell nucleus and induce the genetic apparatus of the cell to be mobilized to synthesize newly expressed gene products (proteins) in response to the initial stimuli.

Cell proliferation, cell-death (apoptosis), chemotaxis, metastases etc. are all controlled at the level of the protein-protein interactions involved. To date no methodology has allowed the quantitative measurement of such interactions as well as the localization of such interactions in a relevant live cellular context.

The subject of this invention is a methodology that allows the quantitative analysis of protein-protein interactions within a living cell in response to stimuli.

The novel methodology of this invention enables: (1) Direct visualization of the molecular architecture of specific cellular responses at the level of the discrete protein-protein interactions that enable such cellular architecture; (2) Direct and quantitative analysis of drug effects on cellular signaling networks in a manner never previously possible; and (3) The creation of quantitative “Pharmacological Profiles” of protein-protein interactions induced in response to stimuli throughout the entire cell; from membrane to nucleus.

The method requires that gene(s) encoding the proteins that encompass a particular signaling pathway of interest are available; preferably as characterized full-length cDNA(s) or less preferably as cDNA libraries. The methodology is not limited, however, to full-length clones and partial cDNA's can also be employed.

For the signaling network to be studied each of the members of selected cDNAs is cloned as a gene fusion in frame (3′ or 5′) with gene fragments that encode a PCA reporter molecule into an appropriate vector such that upon transfection into an appropriate cell line the encoded protein that is subsequently expressed in the cell carries a protein (polypeptide) fragment either at the amino or carboxy terminus of the protein encoded in the original cDNA selected.

Each member of the signaling network is transfected in a gene-by-gene array or matrix, i.e.:

| | Gene A | Gene B | Gene C | Gene D | Gene E | Etc. |
|---|---|---|---|---|---|---|
| Gene A | | | | | | |
| Gene B | | | | | | |
| Gene C | | | | | | |
| Gene D | | | | | | |
| Gene E | | | | | | |
| Etc. | | | | | | |

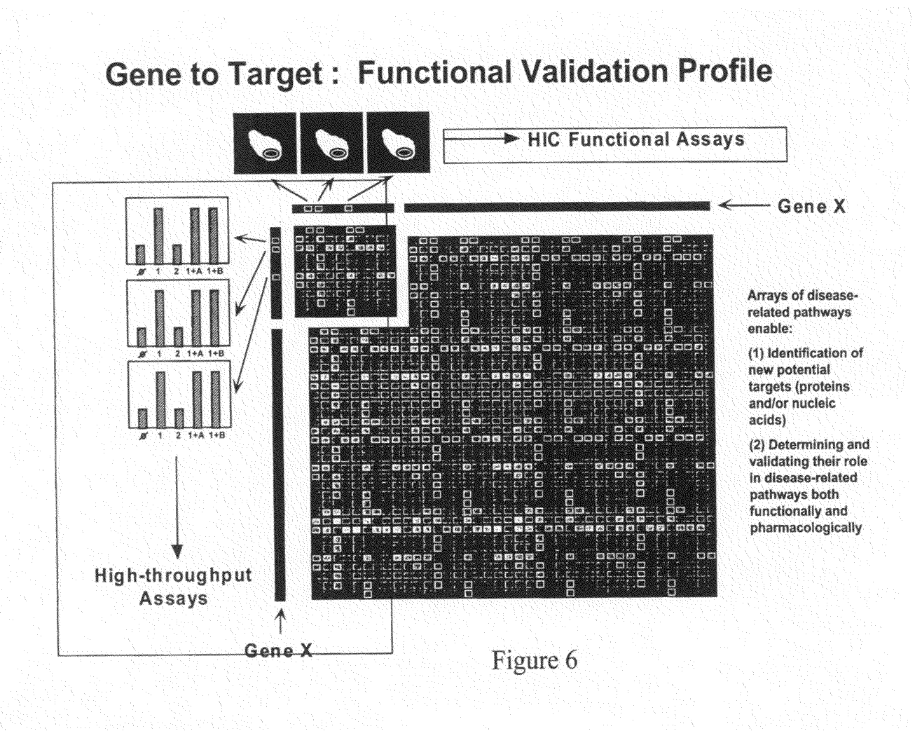

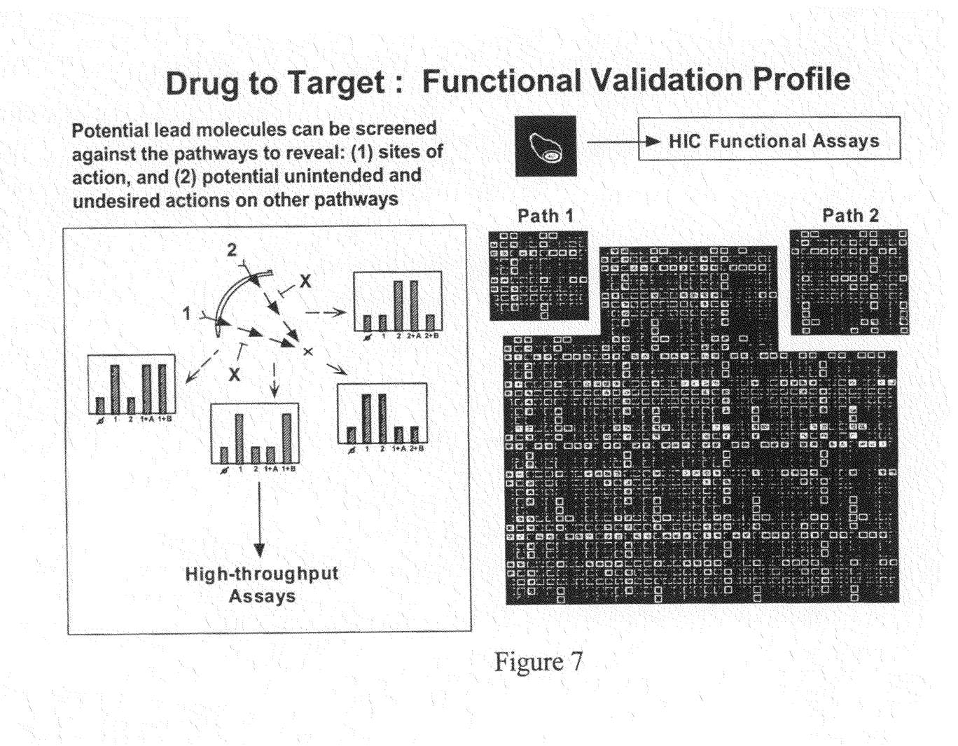

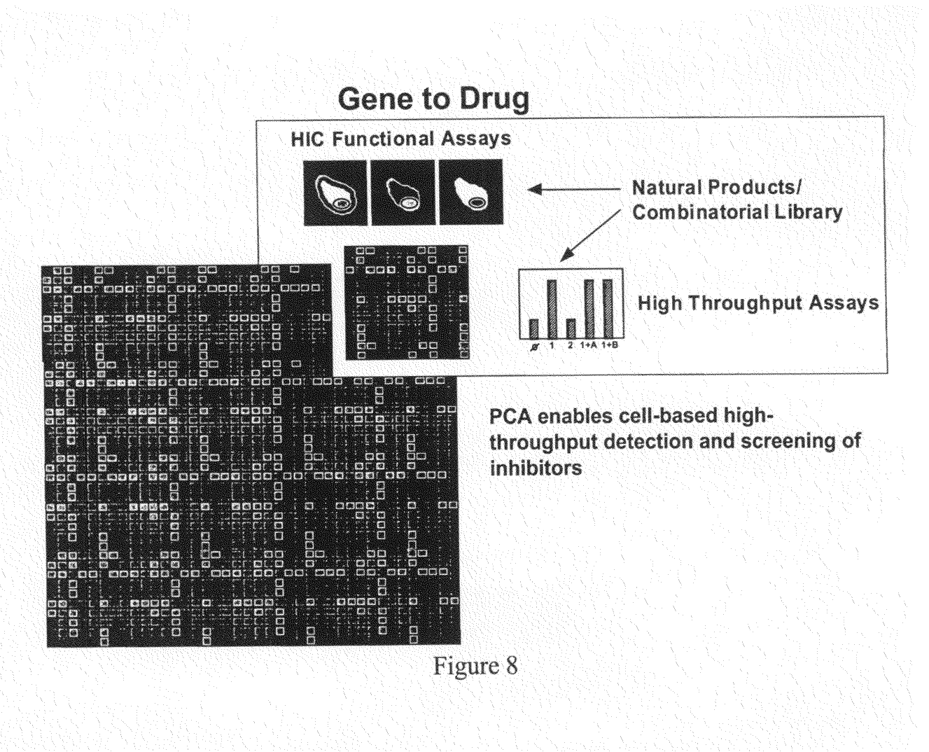

The results of such an interaction map can be depicted as a gene-by-gene matrix as shown in FIGS. 6-8. Pair-wise interaction mapping is achieved using polypeptide reporter tags derived from an enzyme that is essential for cell survival such that cells grow when an interaction occurs (survival selection). Following selection the resultant cells can be studied employing a small molecule inhibitor of the same enzyme that binds stoichiometrically to the reconstituted enzyme and reports the number of protein-protein complexes as well as the physical localization within the cell. The preferred enzyme for this approach is murine dihydrofolate reductase (mDHFR) since this reporter allows survival selection to be carried out in DHFR −/− mammalian cells (CHO) as well as fluorescent visualization employing methotrexate-fluorescein (MTX-Fl); however, other PCA reporters enabling screening, quantitation and localization can also be used.

Quantitation of the number of protein-protein complexes for each pairwise interaction in the cell is achieved, for the DHFR PCA, utilizing fluorophore labeled-MTX and fluorescence detection in a microtiter plate fluorimeter. Stimulation and Inhibition of the interactions is monitored after treatment with stimulus (hormone, ligand, serum, growth-factor etc.). This allows the profiling of responses for each interacting pair of the pathway and also allows the site of intervention of agonists/antagonists within each pathway as well as locating cross-talk points between different signaling pathways as embodied in the attached publication.

The present invention provides a strategy for genome-wide mapping of biochemical pathways using PCA which would entail first a screening step; a simple assay to detect protein-protein interactions among potential partner proteins, followed by the generation of a functional validation profile. Such a profile would consist of two types of data. First, a biochemical network of interest should be perturbed by specific stimuli or inhibitors, for example hormones, drugs or nutrients. By the same reasoning, interactions between component proteins of the pathway should be perturbed by these reagents and a pattern of responses or “pharmacological profile” observed by PCA that is consistent with the response of the pathway or network studied.

In a first embodiment of the invention, we describe screening of protein-protein interactions in the RTK/FRAP pathways. The first step in carrying out the invention was to screen proteins that are known to be involved in RTK- and FRAP-mediated signaling for interactions amongst each other, using the simple DHFR survival-selection PCA. Prokaryotic and eukaryotic DHFR is central to cellular one-carbon metabolism and is absolutely required for cell survival. The principle of the survival assay is that cells simultaneously expressing complementary fragments of DHFR fused to interacting proteins or peptides will survive in media depleted of nucleotides, only if the proteins interact and then bring the complementary fragments of DHFR into proximity where they can fold and reassemble into active enzyme. For survival selection studies, we used CHO DUKX-B11 (DHFR⁻) cells stably co-transfected with various combinations of the fusions. Co-transfectants were selected for survival in nucleotide-free medium (selection for DHFR activity). The survival selection assay is extremely sensitive; we have previously demonstrated that only 25 to 100 molecules of reconstituted DHFR per cell are necessary for cell survival (4). DHFR+ cell lines can be used in a recessive selection strategy (6) or the screening for protein interactions could be achieved with the fluorescence assay. However, for the fluorescence studies as further described below, it was more convenient to work with homogenous clonal population of cells expressing exogenous proteins at low levels.

Protein-protein interactions were tested with three variations of the protein DHFR fragment fusions. First, except in specific cases, we tested the same interactions with fusions of the test proteins at either the N- or C-terminus of DHFR fragments. We tested these variants because, not knowing the structures of these proteins, it would not be possible to predict whether the complementary DHFR fragments could be brought into proximity because the individual C- or N-termini of the interacting test proteins are too far from each other. A second variation tested was what we call a fragment-swapping control. We reasoned that if an interaction is observed with one protein-fragment configuration (e.g. X-F[1,2] and Y-F[3]), swapping proteins and fragments should give the same result (i.e. Y-F[1,2] and X-F[3]). These controls could preclude a remote source of false-positive signals. It is possible that a specific protein-fragment fusion could induce fragment complementation in the absence of interaction with a partner protein-fragment fusion, by some alternative mechanism. For example, if a protein were to interact with the fused DHFR fragment in a way that induced the independent folding of the fragment, the folded fragment might act as a template for binding to the complementary fragment, independent of interaction between the test proteins. Such spontaneous complementation is a problem inherent to another approach for measuring protein-protein interactions based on β-galactosidase subunit complementation, in which, the pre-folded subunits always interact to some extent, leading to a false-positive signal of an amplitude dependent on protein expression levels (10). This problem does not occur with the DHFR PCA because the fragments are incapable of folding independently (6, 11). Finally, we also tested “kinase-dead” forms of some of the protein kinases studied here. These mutants, by acting as “substrate traps”, are thought to bind with higher affinity to their substrates.

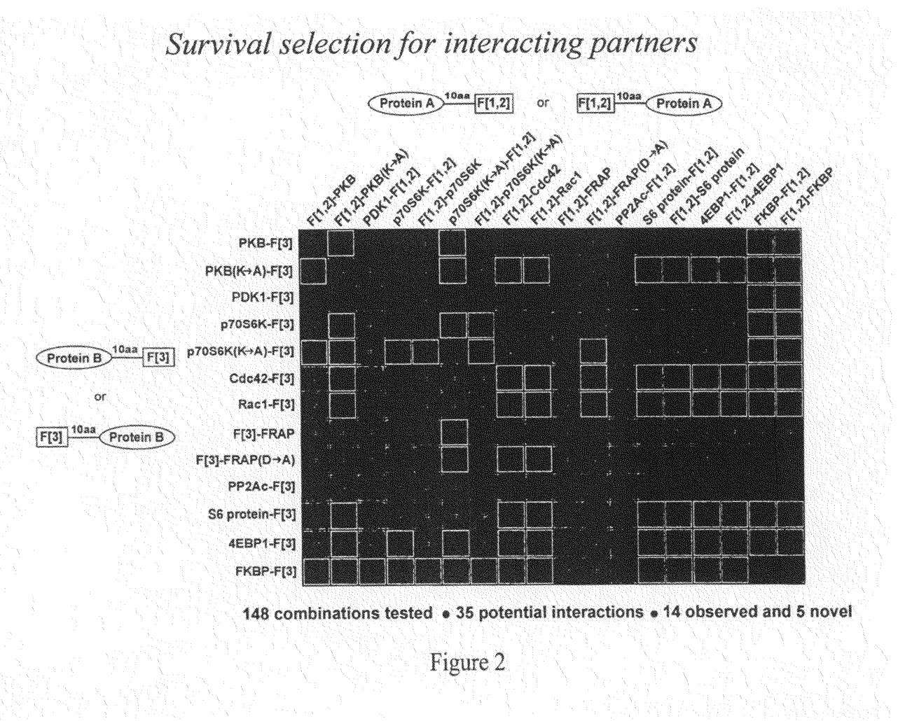

A total of 148 combinations of 35 different protein-protein interactions in the RTK/FRAP signal transduction pathways were tested against each other (FIG. 2). In all cases, full-length protein-DHFR fragment fusions were expressed. Of the 35 interactions tested, 14 corresponded to interacting partners. Nine of these interactions have been previously identified. However, we were surprised to find 5 additional interactions that have not been previously reported or have only been inferred, based on indirect evidence. We discuss these in detail below. Growth rates for colonies of clones expressing differently oriented fusions were not significantly different, suggesting that the linker length was sufficiently long to allow proteins to interact and for the DHFR fragments to be brought into proximity to fold/reassemble. We used a flexible linker peptide of 10 amino acids between the proteins and DHFR fragments, allowing us to probe interactions over distances of 80 Å (˜4 Å per peptide bond×10 amino acids×2 linkers: 1 per fusion). In the cases where we tested the substrate-trapping mutants of protein kinases, we observed no difference in the growth rates of these compared to the wild type, active kinases. The dissociation constants for kinase/substrate interactions are low (˜10 nM to 10 μM) (12) and these values are well within the range of detection of the DHFR survival PCA (4).

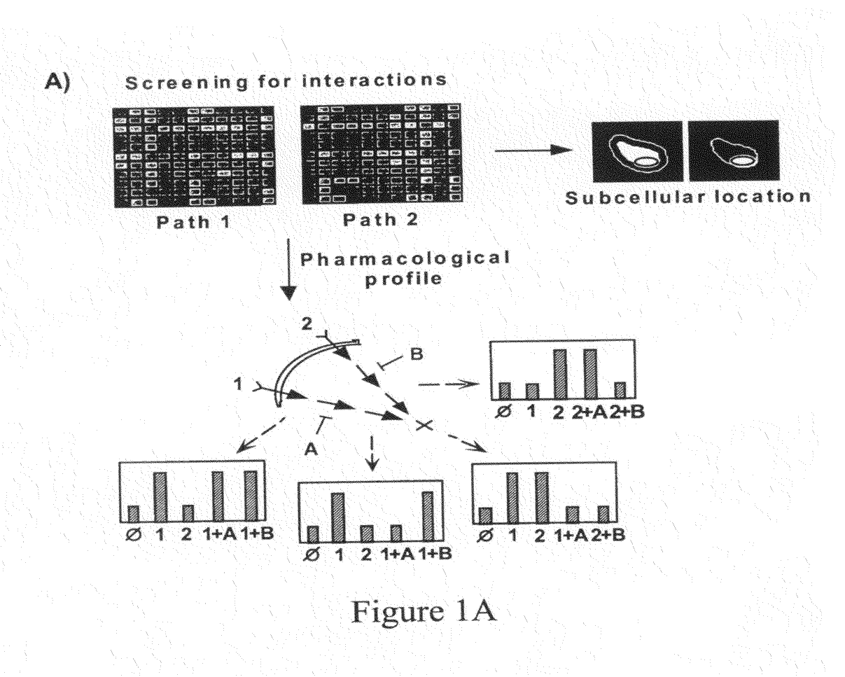

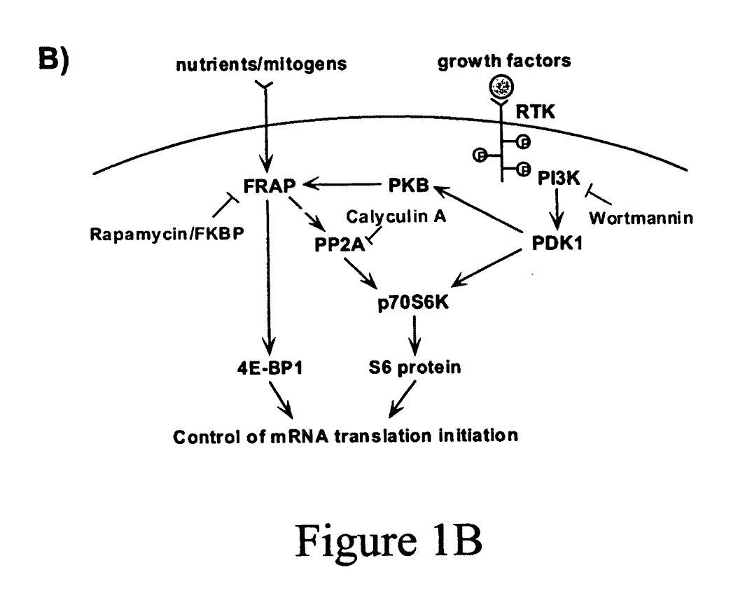

In a second embodiment, the invention provides a means for identifying pharmacological profiles and cellular location of interacting proteins. As discussed above, an important object of the invention was to demonstrate that the DHFR PCA could be used to simultaneously screen and functionally validate protein-protein interactions. Two functional validation experiments were envisioned (FIG. 1A): (1) experiments that would allow the measurement of the effects of pathway-specific stimulators and inhibitors on individual protein-protein interactions in a signaling cascade and (2) experiments that would allow for the unambiguous determination of the physical location of the interaction. It is possible to obtain both types of information with a fluorescence DHFR PCA. The basis of this assay is that complementary fragments of DHFR, when expressed and reassembled in cells, will bind with high affinity (K_{d}=540 pM) to fluorescein-conjugated methotrexate (fMTX) in a 1:1 complex. fMTX is retained in cells by this complex, while the unbound is actively and rapidly transported out of the cells (4, 5). Thus, the fluorescence signal measured in an intact living cell is a direct stoichiometric measure of the number of molecules of reconstituted DHFR and by inference the number of interacting protein complexes. Fluorescence can be measured by any standard spectroscopic technique, including fluorescence-activated cell sorting (FACS) or spectroscopy. The locations of the complexes within a cell can be monitored by simple florescence microscopy and also by automated imaging systems. Since the observed fluorescence arises from 1:1 complexes of fMTX and reconstituted DHFR, the location of the fluorescence in the cell represents the location of the interacting protein complexes.

Insulin and serum-induced signaling have been studied in detail in CHO cells (13) and biochemical analyses of insulin receptor-mediated and serum-induced RTK/FRAP pathways are well documented (for review, see (14)). We restricted the pathway inhibitors used in these studies to three small molecules; wortmannin, which inhibits PI-3K; rapamycin, a specific inhibitor of FRAP; and calyculin A, an inhibitor of the serine/threonine phosphatase PP2A. We chose these inhibitors because their sites and mechanisms of action are well known and because they act at key points in the pathways studied (FIG. 1B). Specifically, wortmannin acts upstream of all of the interactions we studied in the RTK pathway, but should have no effects on interactions downstream of FRAP. In contrast, rapamycin should affect all interactions downstream of FRAP. Both drugs would be predicted to inhibit protein-protein interactions that are downstream of both the RTK and FRAP pathways (e.g. p70S6K with S6 protein, FIG. 1B). Calyculin A is a specific inhibitor of PP2A and thus should interfere with the interactions between PP2A and its substrates.

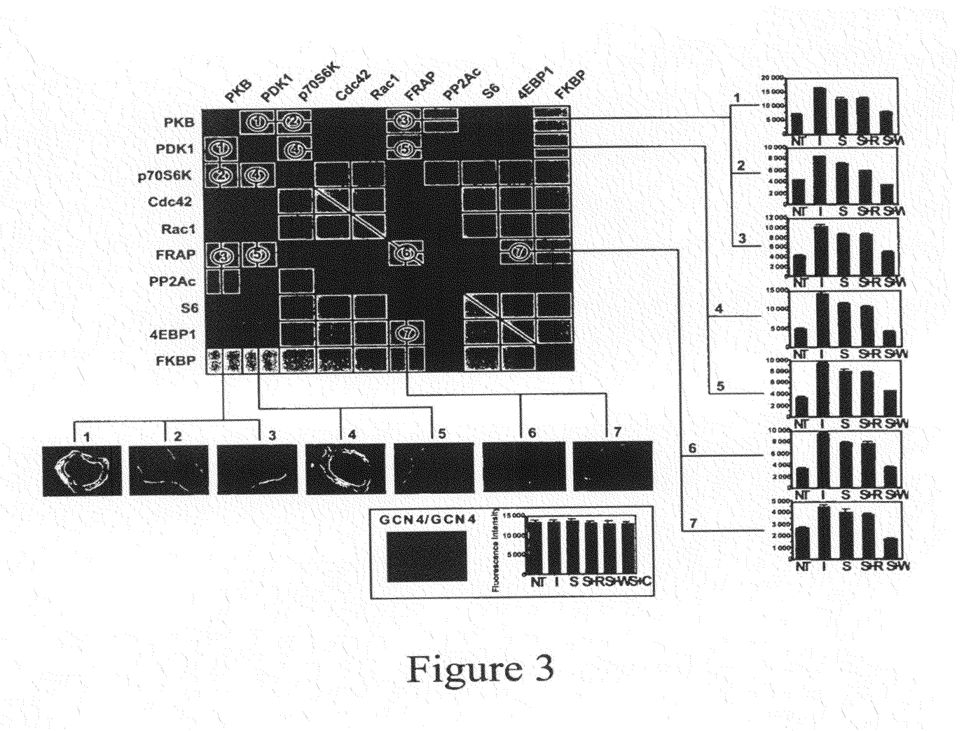

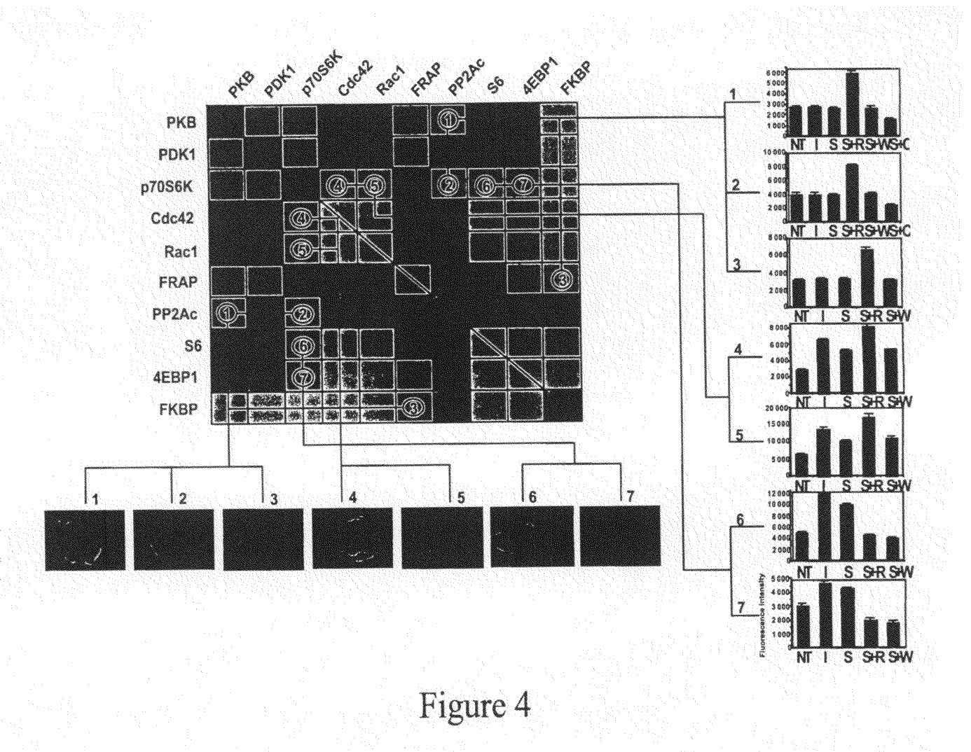

Fluorometric experiments were performed as described in the Examples, using the stable cells derived from the survival-selection screening described above. The fluorescence spectroscopy results on living cells fell into two categories, based on distinct pharmacological profiles: (1) insulin and serum-stimulated and wortmannin-inhibited interactions (FIG. 3) and (2) rapamycin-sensitive interactions (FIG. 4). As we expected, all interactions downstream of insulin or serum-activated signal transduction pathways responded to these stimuli and all were blocked by wortmannin.

For example, we observed a direct interaction between PDK1 and PKB. PDK1 has been identified as a specific PKB kinase (for review see (15, 16)). Further, fluorescence microscopy results showed that the interaction between PDK1 and PKB occurs exclusively at the plasma membrane (FIG. 3:1). It has been proposed that membrane localization of both enzymes is required for PKB phosphorylation by PDK1, via binding to PIP₃ through pleckstrin homology (PH) domains of both kinases. PDK1/PKB interaction is an early step in RTK pathways and therefore the membrane association of the complex is consistent both with known molecular mechanisms of localization and with the pathway hierarchy. p70S6K is also a substrate of PDK1 (17, 18) and predictably, the pharmacological profile and cellular locations of this interaction were identical to those of PDK1/PKB (FIG. 3:4). We also observed a novel, direct interaction between PKB and p70S6K with the same pharmacological profile as PDK1/PKB, but with a cytosolic distribution (FIG. 3:2). This interaction has been suspected but never demonstrated before and PKB has not been shown to act as a p70S6K kinase in vitro (19).

Recent studies suggest that the RTK and FRAP pathways are not entirely independent and our results are consistent with the view that there is considerable cross talk between the two pathways. For instance, we observed not only a direct and previously known interaction between PKB and FRAP (20) (FIG. 3:3), but also a novel interaction between PDK1 and FRAP (FIG. 3:5). Both showed identical pharmacological profiles and cellular locations. We also observed insulin- and serum-induced homodimerization of FRAP (FIG. 3:6). Since homodimerization is induced by insulin and serum and inhibited by wortmannin, FRAP homodimerization might be activated by PDK1 and/or PKB. The observed interaction between FRAP and 4EBP1 is consistent with studies showing that FRAP can directly phosphorylate 4EBP1 in vitro (21, 22) (FIG. 3:7). However, this interaction has the same pharmacological profiles as others in the RTK pathway, again suggesting that RTK pathways may mediate this interaction directly through FRAP.

Rapamycin-sensitive protein-protein interactions showed three distinct pharmacological profiles (FIG. 4). The first profile (1, 2 and 3) consisted of interactions insensitive to insulin, serum and wortmannin, but enhanced by rapamycin. These include the well-known, rapamycin-induced cytosolic interaction between FKBP and FRAP (7, 23) (FIG. 4:3); the recently described interaction of the serine/threonine phosphatase PP2A with p70S6K (FIG. 4:2); and a novel, though inferred interaction of PP2A with PKB (24) (FIG. 4:1). Moreover, the complexes were partially inhibited by the PP2A-specific inhibitor calyculin A, suggesting that the interaction occurs in part between the catalytic site of PP2A and substrate sites on p70S6K and PKB. Evidence from genetic and biochemical studies in yeast and mammalian cells suggests that the actions of FRAP are mediated indirectly through PP2A. Specifically, it has been proposed that FKBP/rapamycin sterically prevents the phosphorylation by FRAP of an inhibitory subunit of PP2A (25, 26) resulting in dissociation of the inhibitory and catalytic subunits of the phosphatase, which thereby allows PP2A to dephosphorylate and deactivate p70S6K. Our results are consistent with this model, placing the interaction of PP2A with p70S6K downstream of the FKBP-rapamycin-FRAP complex. A similar mechanism acting on PKB is also supported by our results.

A second profile supports a model for membrane anchoring of p70S6K at the membrane surface by the Rho family GTPases Cdc42 (FIG. 4:4) and Rac1 (FIG. 4:5) (27). Both of these interactions could be seen to occur at the plasma membrane. p70S6K contains no known membrane anchoring domains and yet interactions with a kinase known to activate it—PDK1—occur at the plasma membrane (FIG. 3:4). The pharmacological profiles were identical for both interactions: rapamycin enhanced the serum-induced association whereas wortmannin had no effect. Our results can be interpreted in the same way as for rapamycin effects on the p70S6K/PP2A interaction: in the presence of rapamycin, PP2A is activated, resulting in hypophosphorylation and translocation of p70S6K to the membrane via direct interactions with Cdc42 and Rac1.

Finally, the downstream interaction of p70S6K/S6 protein is affected by all drugs and stimulants, defining a third profile (FIG. 4: 6) and a point of convergence of the two pathways (“X” in FIG. 1A). However, we also observed a novel interaction between p70S6K and 4EBP1 which has the same pharmacological profile and cytosolic location as p70S6K/S6 (FIG. 4:7). There is no evidence that 4EBP1 is a substrate of p70S6K in vitro; however, 4EBP1 has been shown to be phosphorylated on multiple residues in vivo and rapamycin prevents phosphorylation of some of these sites (28, 29). Further, it has been shown that dephosphorylation of 4EBP1 in response to rapamycin is mediated indirectly by PP2A (30). Our results suggest that the direct link between PP2A and 4EBP1 may be dephosphorylation and inactivation of p70S6K by PP2A.

In a further embodiment, we applicants recognized the potential mechanism by which Rapamycin, a potent immunosuppressive drug also acts as an anticancer agent, inhibiting lymphoid or tumor cell proliferation. rapamycin has been thought to inhibit cancer cell growth through interference with events required for G₁ to S phase progression in cycling cells (1-3). A rapamycin analogue CCI-779; Wyeth-Ayerst is in Phase I clinical trials in cancer patients in the United States and Europe. The applicants' results suggest a more direct, mechanistic link through the modulation of PKB activity by the protein serine/threonine phosphatase PP2A The molecular pharmacology underlying the early biochemical effects of Rapamycin are understood in considerable detail. Rapamycin treatment triggers the rapid dephosphorylation and inactivation of p70S6K in mitogen-stimulated cells (4-8). p70S6K appears to require continuous signaling through FRAP to both achieve and maintain the activated state. Recent findings suggest that FRAP phosphorylates and suppresses the activity of a type 2A protein phosphatase (PP2A) bound directly to p70S6K. Hence Rapamycin treatment may inactivate p70S6K by removing the FRAP-dependent inhibitory constraint on PP2A. A second downstream protein targeted by FRAP is the translational repressor protein, PHAS-1, also called 4E-BP1. PHAS-1 represses translation initiation through association with the elf-4F complex. The phosphorylation of PHAS-1 induced by hormonal stimuli is strongly inhibited by rapamycin implicating that FRAP is directly responsible for the phosphorylation of PHAS-1 in intact cells. However, accumulated genetic and pharmacological evidence places FRAP downstream of PI3K and the PI3K-regulated kinase, Akt (PKB) in growth factor-stimulated cells. In addition, results reported here, clearly demonstrate that PKB is a direct target of PP2A and that the interaction of PP2A with PKB is positively modulated by rapamycin's actions on FRAP. Thus PKB may be regulated by rapamycin in exactly the same manner as p70S6K is controlled. As described below, rapamycin induces apoptosis in some cancer cells. Since decreased activity of PKB is directly associated with this phenotype, whereas inhibition of translation-initiation is not, it is more likely that rapamycin-induced inhibition of PKB activity via PP2A activation is a more likely mechanism of action in inducing apoptosis in cancer cells. These data indicate a novel anticancer drug strategy focused on PP2A (and its isomers and subunits) as a target, for example via activation of the catalytic subunit of PP2A, or via inhibition of the inhibitory subunit of PP2A.

The decision between survival and death is an important aspect of cellular regulation during development and malignancy. Central to this regulation is the process of programmed cell death or apoptosis. A variety of signaling cascades have been implicated in modulation of apoptosis including the phosphatidylinositol 3′ kinase (PI3′K) pathway. Activation of PI3′K, a lipid kinase, protects against apoptosis, whereas its inhibition enhances apoptosis.

The protective effects of PI3′K in mammalian cells have been linked to its activation of a series of phosphatidylinositol-dependent kinases (PDK's), including PDK-1, PDK-2 and protein kinase B (PKB or PKB). PKB is a mitogen-regulated protein kinase involved in the protection of cells from apoptosis, the promotion of cell proliferation and diverse metabolic responses. The activation of PKB by PI3′K is mediated through the binding of the PI3′K phosphorylated phosphatidylinositol signaling phospholipids PI (3,4)P2 and PI(3,4,5)P3 to the pleckstrin homology (PH) domain of PKB.

Binding of the PH domain of PKB to membrane PI(3)P's causes the translocation of PKB to the plasma membrane bringing it into contact with membrane-bound PKB-kinases [phosphatidylinositol-dependent kinase-1 and -2 (PDK1 and 2)], which phosphorylate and activate PKB (Thr 308 and Ser 473).

PKB is a proto-oncogene that inhibits apoptosis by phosphorylating a number of downstream targets. This includes phosphorylation of Bad (Ser112 and Ser136): The phosphorylation of the pro-apoptotic protein Bad results in its cytosolic sequestration by the tau form of 14-3-3 proteins which prevents its binding to the cell survival factor Bcl-XL at intracellular membrane sites and promotion of cell-death. Thus, inhibition of PKB activation induces cancer cell apoptosis.

Recent studies employing immunological techniques and ‘in vitro’ kinase assays coupled with phosphopeptide specific antibodies has substantiated that FRAP is a downstream target of one or more of the PDK's in insulin- or growth factor-stimulated cells (see for example: A. Sekulic et. al., Cancer Research, 2000, 60, 3504-3513). Since the anticancer as well as the immunosuppressive activity of rapamycin may be related to the disruption of the FRAP-dependent limb of the PI3K-PKB signaling pathway during G1 phase of the cell cycle the availability of a mammalian cell based assay to screen for modulators of such a signaling pathway could lead to the identification of novel therapeutic agents that may be effective in treating a number of diseases associated with dysregulation of PI3K-PKB and FRAP signaling.

It has been reported that PKB is overexpressed in some mammalian pancreatic, prostate, ovarian and breast cancers as well as glioblastomas and leukemia. It is also known that defects in the insulin signaling pathway are associated with mutations in PKB. Such diseases may include hyperglycemia, particularly non-insulin dependent diabetes mellitus (NIDDM); by related disorders which may include, obesity, hypertension, hypercholesterolemia, hypertriglyceremia, etc.

Protein-Protein Interactions identified by ‘in vivo’ mapping of signal transduction pathways inducing activation of the 70 kD S6 ribosomal protein serine/threonine kinase (p70S6K) have been identified by protein complementation assays in live cells these are: PDK1: PKB, PDK1: p70S6K, PKB:p70S6K, PKB:FRAP, PDK1: FRAP, FRAP:4EBP1, PP2A:p70S6K, PP2A:PKB, P70S6K:Rac1, P70S6K:Cdc42, P70S6k:4EBP1. PCA can be used to construct assays for novel small-molecule inhibitors or activators of these proteins, in order to identify novel anticancer and immunosuppressive drugs from small-molecule, natural product or other compound libraries.

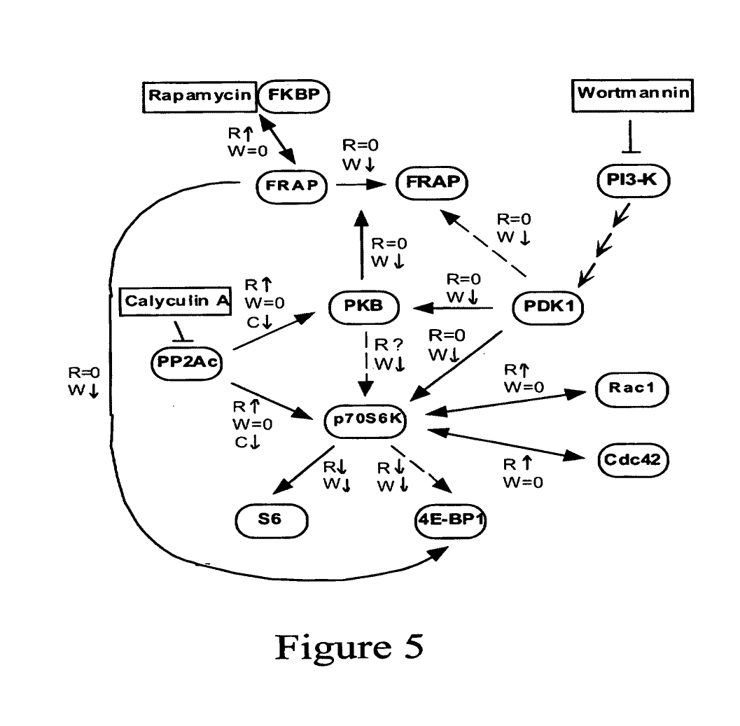

We have demonstrated a simple and sensitive assay to select clonal populations of cells in which a specific interacting pair of proteins is expressed. Further, the pharmacological profiles and cellular location of interactions allowed us to “place” each gene product at its relevant point in the pathways. From the results of our analysis a map of the organization of the RTK/FRAP network emerges, which is consistent with existing models for the organization of the pathway, but includes novel interactions revealed that should be explored in more detail (FIG. 5). The ability to monitor a network of protein interactions in living cells containing components of the underlying pathway studied here revealed hidden connections, not observed before, in spite of intense scrutiny of this network. Further, such analysis is not limited to a specific cell type; we have already demonstrated the utility of PCA strategies in bacteria and mammalian cells (3-6). The results presented here demonstrate that PCA has the features necessary for a general protein function validation and pathway mapping strategy.

### EXAMPLES

The present invention is illustrated by the following Examples, but should not be construed to be limited thereto.

### Example 1

DNA constructs. The full-length cDNAs encoding PKB and PKB(K→A), PDK1, p70S6K and p70S6K(K→A), S6 ribosomal protein, FRAP and FRAP(D→A), 4EBP1, FKBP, the alpha catalytic subunit of PP2A, and the GTPases Cdc42hs and Rac1 were amplified by PCR and subcloned into the eukaryotic expression vector pMT3 (9), in 5′ or 3′ of the F[1,2:F31S], which we call F[1,2], and the F[3] fragment of dihydrofolate reductase (DHFR) (4). F[1,2] corresponds to amino acids 1 to 105 and F[3] to amino acids 106 to 186 of murine DHFR. In all cases, a 10 amino acids flexible linker consisting of (Gly.Gly.Gly.Gly.Ser)₂ was inserted between the cDNA and the DHFR fragments. The ZIP-F[1,2] and ZIP-F[3] constructs (described in: (4)) consist of fusions with GCN4 leucine zipper-forming sequences.

### Example 2

DHFR survival selection assay. CHO DUKX-B11 (DHFR⁻) cells were split 24 hours before transfection at 8×10⁴ in 12-well plates in α-MEM (Life Technologies) enriched with dialyzed fetal bovine serum (FBS; Hyclone) and supplemented with 10 μg/ml of adenosine, desoxyadenosine and thymidine (Sigma). Cells were transfected using Lipofectamine reagent (Life Technologies) according to the manufacturer's instructions. 48 hours after the beginning of the transfection, cells were split at approximately 5×10⁴ in 6-well plates in selective medium consisting of α-MEM enriched with dialyzed FBS but without addition of nucleotides. Cells were observed, for the appearance of colonies, over a period of 5 to 21 days after incubation in selective medium. Only cells expressing fused interacting partners gave rise to colonies. A few surviving colonies were isolated for each transfection by trypsinizing in cloning cylinders and grown individually up to confluence.

### Example 3

Fluorometric analysis. CHO DUKX-B11 cells stably expressing interacting proteins fused to DHFR fragments were split at 2×10⁵ in 12-wells plates in α-MEM (Life Technologies) enriched with dialyzed FBS (Hyclone) and incubated for 24 hours. Cells were washed with α-MEM and serum starved (0.5% dialyzed FBS) in α-MEM containing 10 μM fMTX (Molecular Probes) for 20 hours. Medium was removed, cells were washed, incubated in α-MEM containing 10 μM fMTX, but without serum, for 3 hours and untreated or treated with 20 μg/ml insulin (Roche Diagnostics) or 15% serum for 30 mins. For the drug treatments, after the 20 hours incubation, cells were pre-treated with 20 nM rapamycin (Calbiochem) or 300 nM wortmannin (Calbiochem) for 3 hours, or 20 nM calyculin A (Calbiochem) for 45 mins., and then 15% serum was added to the samples for 30 mins. For all the samples, medium was removed and the cells were washed and reincubated for 15 mins. in α-MEM (without fMTX), with addition of drugs, insulin or serum in corresponding samples, to allow for efflux of unbound fMTX. The medium was removed, cells were washed one time with PBS (phosphate-buffered saline) and gently trypsinized. Plates were put on ice and 100 μl of cold PBS was added to the cells. The total cell suspensions were transferred to 96-well white microtitre plates (Dynex) and kept on ice prior to fluorometric analysis (Perkin Elmer HTS 7000 Bio Assay Reader). Afterward, the data were normalized to total protein concentration in cell lysates (Bio-Rad protein assay).

### Example 4

Fluorescence microscopy. COS cells were grown on 18 mm glass cover slips to approximately 2×10⁵ in DMEM (Life Technologies) enriched with 10% cosmic calf serum (CCS; Hyclone) in 12-well plates. Cells were transiently co-transfected with different combinations (as indicated) of the pMT3 plasmid harboring the full-length cDNAs fused via 10 amino acid linkers to F[1,2] or F[3], using Lipofectamine (Life Technologies). 24 hours after transfection, fMTX (Molecular Probes) was added to the cells at a final concentration of 10 μM. After an incubation of 20 hours, medium was removed and cells were washed and reincubated for 15 mins. in DMEM enriched with 10% CCS, to allow for efflux of unbound fMTX. The medium was removed and cells were washed 2 times with cold PBS and finally mounted on glass slides. Fluorescence microscopy was performed on live cells with a Zeiss Axiophot microscope (objective lens Zeiss Plan Neofluar 100×/1.30).

### REFERENCES

1. Fields, S. & Song, O. (1989) Nature 340, 245-6.
2. Uetz, P., Giot, L., Cagney, G., Mansfield, T. A., et al. (2000) Nature 403, 623-627.
3. Pelletier, J. N., Campbell-Valois, F. & Michnick, S. W. (1998) Proc Natl Acad Sci USA 95, 12141-12146.
4. Remy, I. & Michnick, S. W. (1999) Proc Natl Acad Sci USA 96, 5394-5399.
5. Remy, I., I. A. Wilson, and S. W. Michnick (1999) Science 283, 990-993.
6. Michnick, S. W., Remy, I., C.-Valois, F.-X., V.-Belisle, A. & Pelletier, J. N. (2000) in Methods in Enzymology (J. N. Abelson, S. D. Emr and J. Thorner, ed) Vol. 328, pp. 208-230, Academic Press, New York.
7. Sabatini, D. M., Erdjument-Bromage, H., Lui, M., Tempst, P. & Snyder, S. H. (1994) Cell 78, 35-43.
8. Brown, E. J., Albers, M. W., Shin, T. B., Ichikawa, K., Keith, C. T., Lane, W. S. & Schreiber, S. L. (1994) Nature 369, 756-758.
9. Kaufman, R. J., Davies, M. V., Pathak, V. K. & Hershey, J. W. (1989) Mol. Cell. Biol. 9, 946-958.
10. Rossi, F., Charlton, C. A. & Blau, H. M. (1997) Proc. Natl. Acad. Sci. USA 94, 8405-8410.
11. Gegg, C. V., Bowers, K. E. & Matthews, C. R. (1997) Protein Sci. 6, 1885-1892.
12. Bhalla, U.S. & Iyengar, R. (1999) Science 283, 381-387.
13. Mamounas, M., Gervin, D. & Englesberg, E. (1989) Proc Natl Acad Sci USA 86, 9294-9298.
14. Avruch, J. (1998) Mol Cell Biochem 182, 31-48.
15. Belham, C., Wu, S. & Avruch, J. (1999) Curr Biol 9, R93-R96.

16. Vanhaesebroeck, B. & Alessi, D. R. (2000) Biochem J 3, 561-576.
17. Alessi, D. R., Kozlowski, M. T., Weng, Q. P., Morrice, N. & Avruch, J. (1998) Curr Biol 8, 69-81.
18. Pullen, N., Dennis, P. B., Andjelkovic, M., Dufner, A., Kozma, S. C., Hemmings, B. A. & Thomas, G. (1998) Science 279, 707-710.
19. Dufner, A., Andjelkovic, M., Burgering, B. M., Hemmings, B. A. & Thomas, G. (1999) Mol Cell Biol 19, 4525-4534.
20. Nave, B. T., Ouwens, M., Withers, D. J., Alessi, D. R. & Shepherd, P. R. (1999) Biochem J 2, 427-431.
21. Brunn, G. J., Hudson, C. C., Sekulic, A., Williams, J. M., Hosoi, H., Houghton, P. J., Lawrence, J., Jr. & Abraham, R. T. (1997) Science 277, 99-101.
22. Gingras, A. C., Gygi, S. P., Raught, B., Polakiewicz, R. D., Abraham, R. T., Hoekstra, M. F., Aebersold, R. & Sonenberg, N. (1999) Genes Dev 13, 1422-1437.
23. Lorenz, M. C. & Heitman, J. (1995) J Biol Chem 270, 27531-27537.
24. Andjelkovic, M., Jakubowicz, T., Cron, P., Ming, X. F., Han, J. W. & Hemmings, B. A. (1996) Proc Natl Acad Sci USA 93, 5699-5704.
25. Di Como, C. J. & Arndt, K. T. (1996) Genes Dev 10, 1904-1916.
26. Jiang, Y. & Broach, J. R. (1999) Embo J 18, 2782-2792.
27. Chou, M. M. & Blenis, J. (1996) Cell 85, 573-583.
28. Heesom, K. J., Avison, M. B., Diggle, T. A. & Denton, R. M. (1998) Biochem J 336, 39-48.
29. Gingras, A. C., Kennedy, S. G., O'Leary, M. A., Sonenberg, N. & Hay, N. (1998) Genes Dev 12, 502-513.
30. Peterson, R. T., Desai, B. N., Hardwick, J. S. & Schreiber, S. L. (1999) Proc Natl Acad Sci USA 96, 4438-4442.

Although the present invention has been described with reference to specific details of certain embodiments thereof, it is not intended that such detail should be regarded as limitations upon the scope of the invention, except as and to the extent that they are included in the accompanying claims.

1-22. (canceled)

23. A method of determining a subcellular location where two or more molecules interact comprising coupling each molecule to a separate part of a reporter system, which parts directly or indirectly provide a detectable signal upon their association, and testing for the subcellular location of said signal.

24. A method according to claim 23 where at least one of said molecules is a protein.

25. A method according to claim 23 where at least two of said molecules are proteins.

26. A method according to claim 23 where all of said molecules are proteins.

27-39. (canceled)
